(19) United States
(12) Patent Application Publication
(10) Pub. No.: US 2010/0200003 A1
(43) Pub. Date: Aug. 12, 2010

(54) HEADGEAR ASSEMBLY AND NOVEL USES THEREOF

(76) Inventor: Bruce Lloyd Armstrong, Howick (NZ)

Correspondence Address: JACOBSON HOLMAN PLLC, 400 SEVENTH STREET N.W., SUITE 600, WASHINGTON, DC, 20004, US

(21) Appl. No.: 12/452,991

(22) PCT Filed: Jul. 25, 2008
(86) PCT No.: PCT/NZ2008/000181

§ 371 Date: Apr. 19, 2010

(30) Foreign Application Priority Data
Jul. 31, 2007 (NZ) 560261

(51) Int. Cl. A61B 19/00

(52) U.S. Cl. 128/869

(57) ABSTRACT

Apparatus for a method of presenting a human head for sleeping or surface support. The method involves the use of the apparatus which is head gear with an exterior relief feature to favour a sleeping or support disposition or to favour a range of such dispositions.

### TECHNICAL FIELD

The present invention relates to headgear assembly and novel uses thereof.

### BACKGROUND ART

There are a number of very “modern day” problems associated with the requirement to now force babies to sleep on their backs to reduce the growing incidence of “cot deaths”.

As babies are sleeping on their backs a baby's head can develop a flat spot or becomes asymmetrical due to the external pressure. This is referred to as Positional Plagiocephaly. Babies with severe plagiocephaly may also have prominent foreheads, misaligned ears, and uneven facial features.

Many babies are born with abnormally shaped heads as a result of the pressure exerted on them during birth, traditionally a variety of sleeping positions and plenty of “tummy time” would correct this naturally within about six weeks, but enforced sleeping on the back interferes with this also.

The key to effective prevention, is as usual early diagnosis of risk and introduction of the preventative measures. Such preventative measures take 2 forms (options A and B).

(A) Repositional measures—Moving a baby from time to time thereby to encourage the, to sleep or lie in a different, or in a variety of different positions, without restraint on the head to spread the load over different parts of the head.

(B) Orthotic measures—Where a cast is taken of the head and a helmet or solid cap is made and the baby wears it 23-24 hours per day for many months, whilst the head grows into the correct shape as forced by the shape of the helmet. Due to the traumatic and mediaeval nature of this treatment it is often not started until the child is 6 months or so and can last from months to a year.

It is therefore an object of the present invention to provide an alternative to options A and B outlined above.

It is a further or alternative object to control head positioning of a human whether prone or not eg, in a pushchair against a surface and/or in a wheelchair against a surface or in a “bucket” type car seat or the like against a surface. Thus it need not be used solely with recumbent sleeping by a human.

### DISCLOSURE OF INVENTION

In an aspect the invention is a head disposition device or assembly comprising or including

headgear adapted to be fitted to a human being, and

at least one (i) relief feature (ii) or relief feature device located, or locatable, or both, on said headgear so as to favour a range of sleeping and/or surface support dispositions of the wearer.

The apparatus is preferably adapted for a human baby.

Preferably the apparatus is for use with sleeping. Optionally it can be used for other purposes eg, non-recumbent surface support whether sleeping or not. Examples include wheelchair, bucket seat and like useage.

Preferably the relief feature or relief feature device is a device that is relocatable.

Preferably said device is bulbous from a substantially flat attachment surface.

Preferably the headgear is soft and/or flexible for conforming to the wearer's head.

Preferably the feature or device of the headgear is relocatable by means of a VELCRO type inter-engagement (preferably flat or near flat surface to flat or near flat surface or between complementary surfaces).

Optionally the headgear is a cap.

It can be a headband, adjustable or otherwise.

Optionally said headgear is fastenable under the chin.

In another aspect the invention is in combination,

headgear engageable to a human head, and

at least one or more shaped features attachable to the exterior of that headgear to control head disposition against a surface.

Preferably the headgear is soft and/or flexible.

Preferably the combination is adapted for use on a human head for recumbent or non-recumbent sleeping or recumbent or non-recumbent support.

In an aspect the invention is a method of presenting a human head for sleeping or surface support, said method comprising or including the use of head gear with an exterior relief feature or device (“feature”) to favour a sleeping or support disposition or to favour a range of such dispositions.

In another aspect the invention is a head disposition device or assembly (eg, whether for sleeping or otherwise) comprising or including

headgear adapted to be fitted to a human being (eg, a baby), and

at least one relief feature or device located or relocatable (preferably relocatable) on said headgear exterior so as to favour a range of dispositions of the wearer (eg, baby) on a support surface. The surface need not be horizontal.

Preferably the feature or device is relocatable.

Preferably the headgear is soft and/or flexible ie, so as not, of itself, to tightly confine, for conforming to its interior shape, the wearer's head.

Preferably the repositioning is by means of a VELCRO type inter-engagement. Other forms of inter-engagement can be utilised (eg, adhesive, sliding, etc).

Preferably said headgear is fastenable under the chin of the baby. Alternatively fit can rely solely on behind head or other lacing adjustment and/or a VELCRO type adjustment.

In a further aspect the present invention consists in, in combination,

headgear engageable to a human (eg, baby's) head, and

at least one or more shaped features attachable to the exterior of that headgear.

Preferably the headgear is soft and/or flexible ie, so as not, of itself, to tightly confine, for conforming to its interior shape, the wearer's head.

In a further aspect the present invention consists in use of a headgear combination or a sleep disposition device or assembly substantially as herein described for any of the purposes herein described.

In a further aspect the present invention consists in a method of presenting a human head (eg, that of a baby) for sleeping or other support (eg, with a view to minimising and/or correcting shape deformity of a baby's head), said method comprising or including the use of head gear and, with, or an exterior relief feature (preferably a repositionable device eg, relief feature or the like attachable to the exterior of said headgear) thereby to favour a sleeping or other support disposition or range of dispositions.

In another aspect the invention consists in a sleeping system to favour particular head disposition or dispositions, the system involving the use of apparatus when sleeping, the apparatus having a raised exterior relief on headwear that discourages sleeping on the raised area.

As used herein the term “and/or” means “and” or “or”, or both.

As used herein the term “(s)” following a noun includes, as might be appropriate, the singular or plural forms of that noun.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

As used herein “headgear” includes caps, helmets, bands, or the like. Preferably fastenable in some way (eg, lacing, VELCRO fasteners, or the like).

As used herein “relief” in respect of feature or device refers to an outstand of some kind discouraging of its directly being under the head ie, more inclined to favour no roll over the outstand from a favoured supported disposition or range thereof.

As used herein “located” can include fitted to or formed in or on the headgear.

Our new headgear proposal aims to improve the effectiveness and reduce the trauma of option A.

As it is only required during sleep it removes the Frankenstein element of the Orthotic device—option B, often referred to as the “Supermarket Effect”.

As the deflector piece is movable and can be made of various shapes and sizes, it can be adapted to cover a range of various positions of the initial problem but can also be moved to prevent merely transferring the problem to another site.

### BRIEF DESCRIPTION OF DRAWINGS

The preferred forms of the present invention will now be described with reference to the accompanying drawings in which

FIG. 1 shows headgear on a human baby's head and showing ready to be attached a relief feature attachable to the exterior of the headgear,

FIG. 2 shows the assembly resulting from the procedure shown in FIG. 1,

FIG. 3 is an alternative cap or helmet form to that shown in FIGS. 1 and 2, the attachable device with a flat attachment surface being shown exploded from the headgear,

FIG. 4 shows one form of assembly arising from the use of a device as in FIG. 3,

FIG. 5 is a view of one form of relief providing device that can be used showing a flat attachment surface, the device being bulbous from such flat surface,

FIG. 6 shows a top view of a device as shown in FIG. 5 showing how the bulbous extension from the flat surface, if desired, can have a modified region thereof,

FIG. 7 is an alternative to the headgear of FIGS. 1 to 4, the device being more in a form of a headband to which a device as shown in, for example, FIGS. 5 and 6 can be attached to any zone receptive to its attachment surface,

FIG. 8 is a cap form that can be used as substitution for the earlier forms of headgear, this cap form having provision for attachment by a strap (not shown) under the chin of a wearer,

FIG. 9 shows the headgear of FIG. 8 fitted to a human baby and showing a chin strap holding the device to the baby and having exploded therefrom an attachable device, and

FIG. 10 shows the full assembly of the apparatus of FIG. 9 with the relief providing feature or device located on the worn item of headgear.

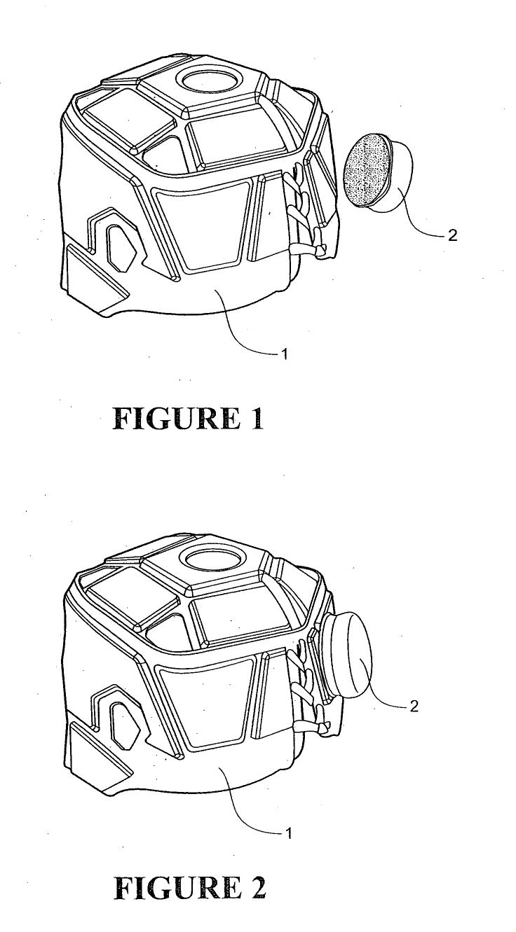

In a preferred form of the present invention as shown is FIGS. 1 and 2 the headgear, which is preferably any suitable soft and compliant headgear shown as 1, is adapted to have selectively attached thereto, as a device, the relief feature as shown as 2.

Preferably there is a hook type inter-engagement (eg, “VELCRO type”) between the relief feature 2 and the headgear 1 although any other suitable inter-engagement form can be utilised (eg, magnetic, adhesive, click fit, or the like).

Preferably the headgear site for device fitment is shaped to complement the fitment region of the device. For example a substantially flat face to a substantially flat face or a convex/concave arrangement or vice versa).

A basic product is a close fitting soft comfortable helmet or cap (cap) made of a sandwich of:
comfortable lining material, with or without various thermal and moisture control properties,
flexible closed cell foam or rubber like materials,
an outer layer of fabric that's outer surface is made as or works as a “loop” in the way of “Hook and loop” fastening materials (eg, VELCRO™).

Making it out of thin foam sandwich, but could be thicker, could be made solely from a single layer of appropriate fabric or something like neoprene.

This headgear is fitted to the infant. It may or may not have some impact protection properties depending on the content of the sandwich. Such properties are not important.

The headgear preferably is sufficiently vented to avoid heat distress of a sleeping wearer.

Supplied with this product is a shape or a number of shapes that are covered on at least one side with “Hook” material. The purpose of the shape or shapes is that when it is stuck to the helmet the baby (or other wearer) cannot lie on the area that is being protected by the shape.

The object must be firm enough to not allow the baby or wearer to sleep on the point or region that is being protected. A suitable rigid form plastic can be used.

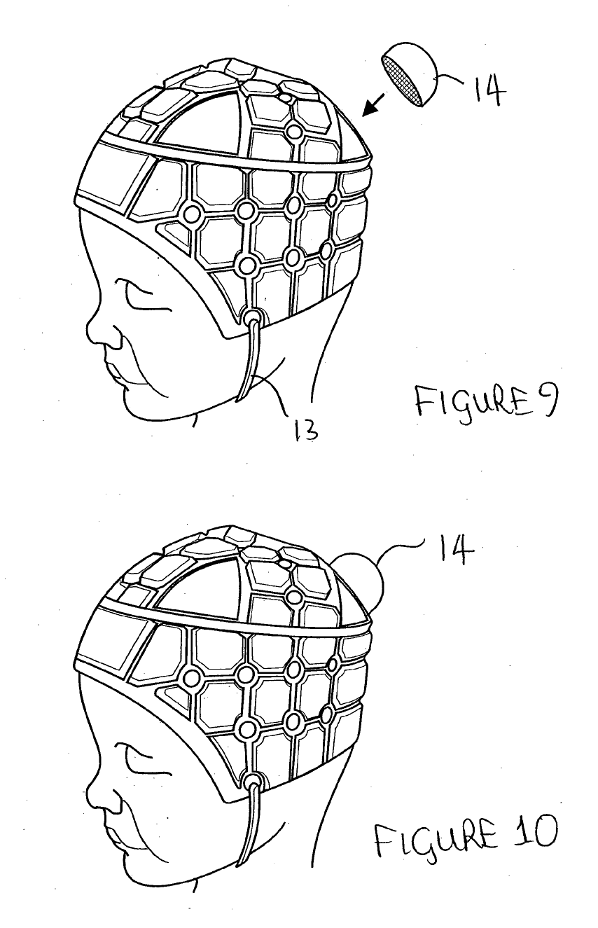

FIGS. 4 through 10 show that it matters not just exactly what is the form of the headgear provided it is reasonably comfortable on the wearer and can be used in conjunction with the intended use whether for sleeping or not.

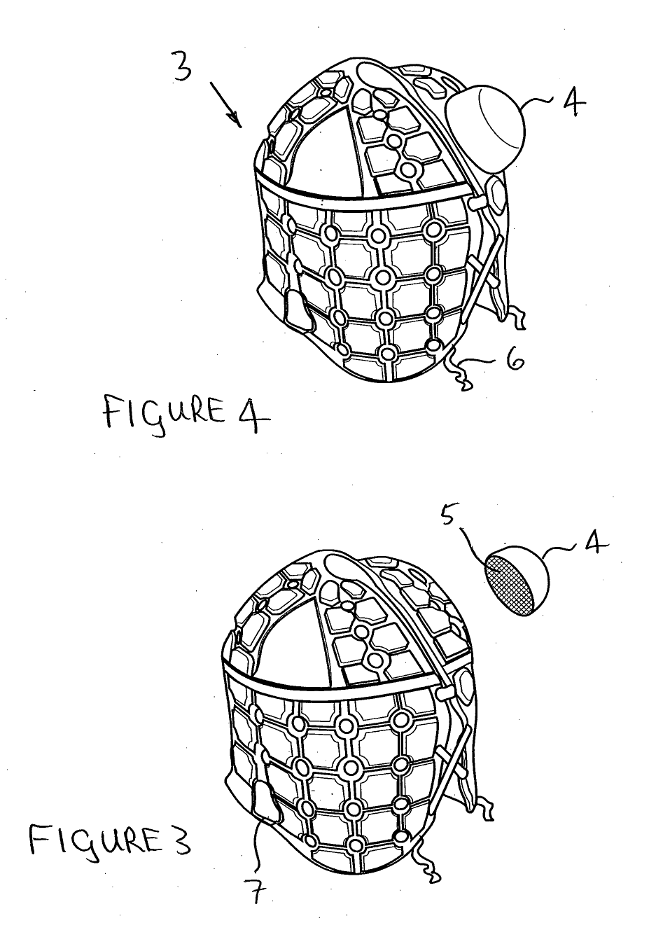

FIGS. 3 and 4 show headgear in the form of a helmet attachable to a range of different head sizes owing to adjustment lacing 6. Located on the headgear 3 is the relief providing device 4 attached at its flat surface 5 by a VELCRO type fitment to a mating VELCRO component fitted surface of the headgear.

If desired the VELCRO interaction can be of a kind that is provided by proprietary paired components, each of which can be adhesively fixed where desired to the headgear 3 and the other of which can be adhesively affixed either as shown as 5 or to the surface 5.

The headgear 3 can have provision for under chin strapping via openings 7 if desired.

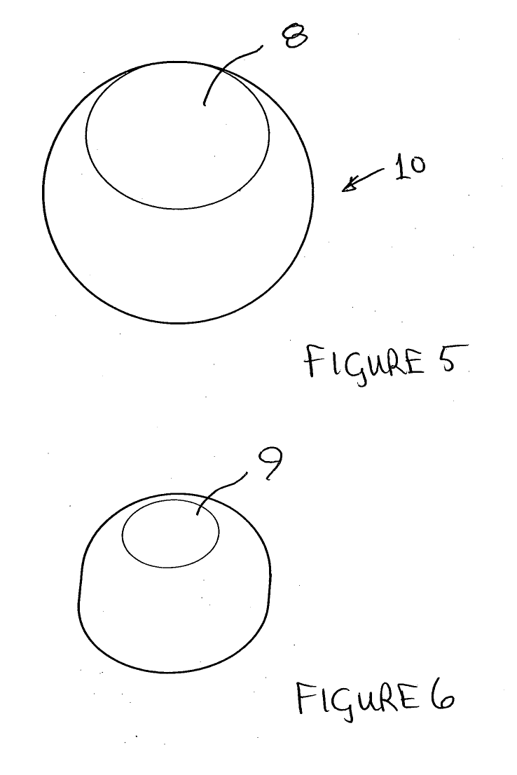

The device can have the flat fitment surface 8 as shown in FIG. 5 to which adhesively a VELCRO attachment component can be adhesively fitted. That would be mated with a like component on the headgear itself.

The reverse side of the arrangement shown in FIG. 5 can have a recess or flat region 9.

Any suitable rigid or compressible material that serves the purpose can be used for the component 10 eg, polyurethane foam, rubber, or the like and can even be a metal such as aluminium. But preferably it is a device which if rolled on is unlikely to hurt the wearer.

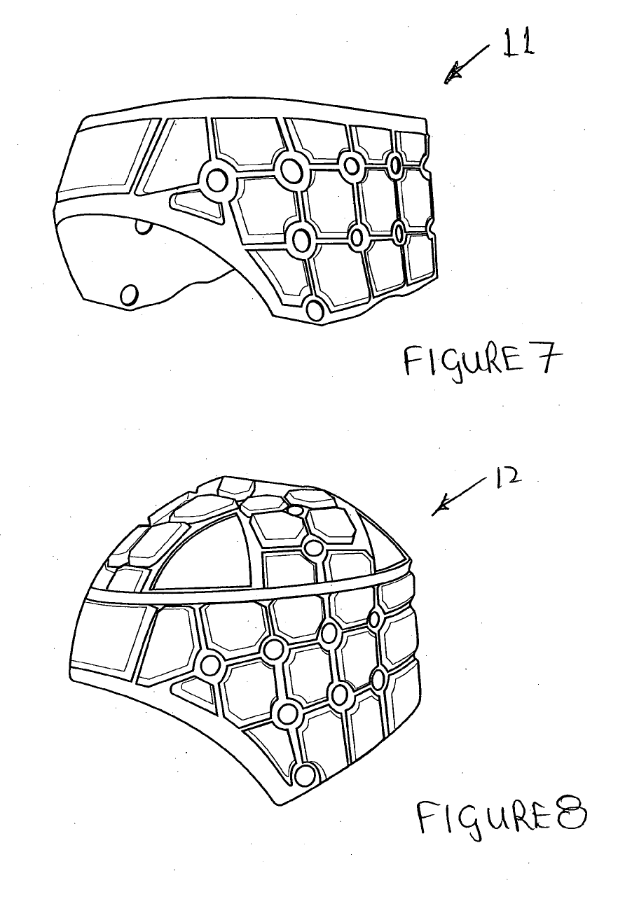

FIG. 7 shows a device 11 in the form of a headband which may, or may not, include lacing of the kind previously shown for headband fitment.

FIG. 8 shows a cap form 12 which can be used in conjunction with under chin strapping 13 and to then have a device 14 located as aforesaid.

The range of products shown could be used for any of the purposes herein before described.

Positional Plagiocephaly (flat Head Syndrome) is an intended market for the invention but can easily be used for any head issues in both children and adults whether recumbent or non-recumbent.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

1. A head disposition device or assembly comprising or including
headgear adapted to be fitted to a human being, the headgear being soft and flexible for conforming to the human being's head, and
at least one relief feature located or relief feature device locatable, or both, on said headgear,
wherein the human being is deterred from lying on the area of the headgear where the at least one relief feature and/or relief feature device is located in use.

2. The apparatus of claim 1 adapted for a human baby.

3. The apparatus of claim 1 for use with sleeping.

4. The apparatus of claim 1 wherein the relief feature or relief feature device is a device that is relocatable.

5. The apparatus of claim 4 wherein said device is bulbous from a substantially flat attachment surface.

6. The apparatus of claim 1 wherein the feature or device of the headgear is relocatable by means of a VELCRO type inter-engagement.

7. The apparatus of claim 1 wherein the headgear is a cap.

8. The apparatus of claim 1 wherein the headgear is a headband, adjustable or otherwise.

9. The apparatus of claim 1 wherein said headgear is fastenable under the chin.

10. (canceled)

11. In combination, headgear engageable to a human head, the headgear being soft and flexible for conforming to the human's head, and at least one or more shaped features attachable to the exterior of that headgear to deter the wearer of the headgear from lying on the area(s) of the headgear where the at least one or more shaped features is attached.

12. A combination of claim 11 adapted for use on a human head for sleeping.

13. (canceled)

14. A method of presenting a human head for sleeping or surface support, said method comprising or including the use of a soft and flexible headgear with at least one exterior relief feature to deter the wearer of the headgear from lying on the area(s) of the headgear where the at least one exterior relief feature is attached.

15. (canceled)

16. The sleeping system to favour particular head disposition or dispositions, the system involving the head disposition device of claim 1 when sleeping, the device having a raised exterior relief on headwear that discourages sleeping on the raised area.
